(19) United States
(12) Patent Application Publication
(10) Pub. No.: US 2022/0242829 A1
(43) Pub. Date: Aug. 4, 2022

(54) PROCESS FOR PREPARATION OF ENZALUTAMIDE

(71) Applicant: Synthon B.V., Nijmegen (NL)

(72) Inventors: Petr MITAS, Blansko (CZ); Oldrich SMEKAL, Blansko (CZ)

(21) Appl. No.: 17/620,314

(22) PCT Filed: Jun. 25, 2020
(86) PCT No.: PCT/EP2020/067854

§ 371 Date: Dec. 17, 2021

(30) Foreign Application Priority Data
Jun. 27, 2019 (EP) 19182954.8

(51) Int. Cl. C07D 233/86

(52) U.S. Cl. CPC C07D 233/86; C07B 2200/13

(57) ABSTRACT

The presented invention relates to a process for preparation of Enzalutamide, compound (1) or a salt or a solvate thereof Formula (1). The presented invention also related to 1,4-dioxane solvate of compound (1).

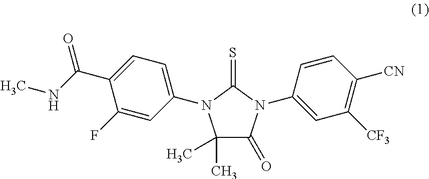 [SMILES: CNC(=O)c1ccc(N2C(=S)N(c3ccc(C#N)c(C(F)(F)F)c3)C(=O)C2(C)C)cc1F]

### BACKGROUND OF THE PRESENT INVENTION

The present invention relates to an improved process for preparation of compound of formula (1). i.e. Enzalutamide or a salt thereof:

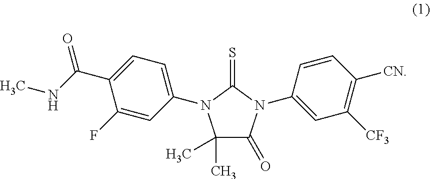 [SMILES: CNC(=O)c1ccc(N2C(=S)N(c3ccc(C#N)c(C(F)(F)F)c3)C(=O)C2(C)C)cc1F]

Enzalutamide, 4-[3-[4-Cyano-3-(trifluoromethyl)phenyl]-5,5-dimethyl-4-oxo-2-thioxoimidazolidin-1-yl]-2-fluoro-N-methylbenzamide is a selective androgen receptor modulator, that is useful for treatment of prostate cancer.

Enzalutamide was first disclosed in WO2006/124118 by The Regents of the University of California. Processes for preparation of Enzalutamide are disclosed in WO2015/063720 by Ranbaxy, WO2015/092617 by Ranbaxy, US20150210649 by Cadila, WO2015/121768 by Ranbaxy, WO2015/154730 by Zentiva, WO2016/005875 by Shilpa, WO2016/038560 by Mylan, WO2016/051423 by Laurus, WO2016/188996, WO2016/188997 both by Olon, WO2016/200338 by Scinopharm, WO2017/081702 by Sun or CN104803918, CN104803919 both by SHANGHAI INST PHARM INDUSTRY.

The disadvantage of the prior art processes are long reaction times or low yields in both reaction intermediates or Enzalutamide.

There is still a need for improved process for preparation of Enzalutamide with relatively short reaction times and good yields and purity of intermediates and Enzalutamide.

### SUMMARY OF THE INVENTION

The presented invention relates to a process for preparation of compound of formula (1) or a salt thereof comprising:

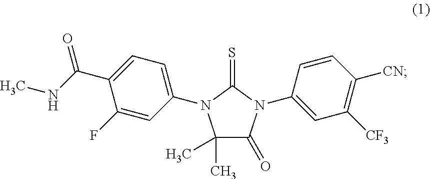 [SMILES: CNC(=O)c1ccc(N2C(=S)N(c3ccc(C#N)c(C(F)(F)F)c3)C(=O)C2(C)C)cc1F]

a. Reacting compound of formula (2) with compound of formula (3) in a solvent under a protective atmosphere, wherein the protective atmosphere is an inert gas streamed above the reaction mixture to provide compound (4):

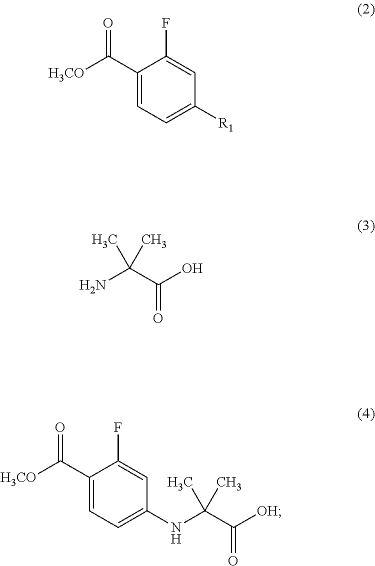

b. Transforming compound (4) into compound (1).

The presented invention further relates to a process for purification of compound of formula (4) comprising:
a. Dissolving compound (4) in a mixture comprising toluene and water and a solvent selected from tetrahydrofurane or dimethylformamide or a mixture thereof;
a. Isolating solid form of compound (4).

The presented invention also relates to 1,4-dioxane solvate of compound of formula (1), a solid form thereof and a process for preparation thereof.

### BRIEF DESCRIPTION OF THE DRAWINGS

FIG. 1: XRPD pattern of solid form of 1,4-dioxane solvate of compound of formula (1).

FIG. 2: XRPD pattern of solid form of compound (1) prepared according to Example 3 or Example 4.

### DETAILED DESCRIPTION OF THE INVENTION

The presented invention relates to a process for preparation of compound of formula (1) or a salt thereof comprising:

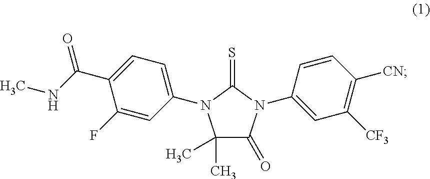 [SMILES: CNC(=O)c1ccc(N2C(=S)N(c3ccc(C#N)c(C(F)(F)F)c3)C(=O)C2(C)C)cc1F]

a. Reacting compound of formula (2) with compound of formula (3) in a solvent under a protective atmosphere, wherein the protective atmosphere is an inert gas streamed above the reaction mixture to provide compound (4):

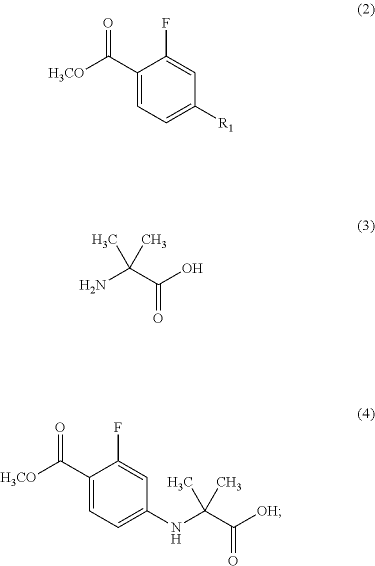

b. Transforming compound (4) into compound (1).

R₁ is a suitable leaving group. The R₁ group can be for example mesylate or tosylate or other alkyl sulfonate or a fluoroalkylsulfonate (such as trifluoromethanesulfonate) or a halogen (such as I or Cl or Br), preferably it is a halogen, more preferably it is Br. Starting compounds (2) and (3) are commercially available.

The solvent in the reaction step a. can be selected from for example dimethylformamide or dimethylacetamide. The reaction is performed in a presence of water. The weight ratio between the solvent and water can be between 1:15 and 1:30, preferably it is between 1:18 and 1:25. The concentration of the compound (2) in the solvent can be between 0.7 g/ml and 1.3 g/ml, preferably it is between 0.9 g/ml and 1.5 g/ml, more preferably between 1 g/ml and 1.3 g/ml. The molar ratio between compound (2) and compound (3) can be between 1:1 and 1:5, preferably it is between 1:1.1 and 1:2, more preferably between 1:1.1 and 1:1.5. The protective gas can be for example argon or nitrogen. The protective atmosphere is streamed above the reaction mixture, i.e. it enters in a first place the space above the reaction mixture and on another place it leaves the space above the reaction mixture by a defined rate. The rate can be between 1 and 100 l/minute (liter/minute), preferably it is between 10 and 100 l/minute, more preferably it is between 30 and 60 l/minute.

Compounds (2) and (3) are mixed with the solvent and a base, that can be selected from an organic base, such as an amine (for example triethylamine, diiso-propylethyl amine) or 1,8-Diazabicyclo[5.4.0]undec-7-ene or 1,5-Diazabicyclo(4.3.0)non-5-ene or 1,4-diazabicyclo[2.2.2]octane or a phosphazene base (such as tert-Butylimino-tris(dimethylamino)phosphorane, tert-Butylimino-tri(pyrrolidino)phosphorane, 2-tert-Butylimino-2-diethylamino-1,3-dimethylperhydro-1,3,2-diazaphosphorine, 1-tert-Butyl-4,4,4-tris(dimethylamino)-2,2-bis[tris(dimethylamino)-phosphoranylidenamino]-2λ5,4λ5-catenadi(phosphazene)) or an inorganic base for example a hydroxide (such as sodium hydroxide or potassium hydroxide or ammonium hydroxide) or a carbonate (such as sodium carbonate or potassium carbonate or lithium carbonate or cesium carbonate) or a hydrogencarbonate (such as sodium hydrogencarbonate or potassium hydrogencarbonate or lithium hydrogencarbonate or cesium hydrogencarbonate) is added. Preferably a carbonate, more preferably K₂CO₃ is used. The molar ration between compound (2) and the base can be between 1:1.5 and 1:5, preferably it is between 1:1.8 and 1:3. To the mixture a salt of Cu⁺ such as CuCl or CuBr or CuI or Cu⁺ acetate is added. The molar ratio between compound (2) and the Cu⁺ salt is between 1:0.18 and 1:0.3, preferably between 1:0.2 and 1:0.25. The mixture is stirred at an elevated temperature for example between 80° C. and 120° C. for between 2 and 4 hours, preferably for between 2 and 3 hours. The reaction progress can be monitored by a suitable analytical technique, e.g. by HPLC or GC. After the reaction is finished, the reaction mixture is cooled to a mixture between 15° C. and 30° C., preferably between 20° C. and 25° C. To the mixture water and tetrahydrofurane are added. The weight ratio between water and tetrahydrofurane can be between 5:1 and 15:1, preferably it is between 7:1 and 10:1. The weight ratio between the tetrahydrofurane and water mixture:solvent used in the step a. can be between 5:1 and 10:1, preferably it is between 6:1 and 8:1. The pH of the mixture is adjusted to 2.0-2.2 using an acid, for example HCl or H₂SO₄ or HBr. To the mixture toluene and tetrahydrofurane (weight ratio between toluene and tetrahydrofurane can be between 1.7:1 and 2.5:1, preferably between 1.9:1 and 2.2:1) are added. The weight ratio between the mixture tetrahydrofurane and toluene:solvent used in the step a. can be between 2:1 and 7:1, preferably it is between 3:1 and 5:1. The phases are separated and the water phase is extracted with toluene. The weight ratio between toluene and solvent used in the step a. can be between 1:0.8 and 1:2, preferably it is between 1:0.9 and 1:1.5. The extraction can be repeated. The mixed organic phases are extracted with water. The weight ratio between water and solvent used in the step a. can be between 1:0.8 and 1:2, preferably it is between 1:0.9 and 1:1.5. The organic phase is concentrated to approx. ¼-⅙, preferably to ⅕ of the original volume. To the rest a mixture comprising toluene, dimethylformamide and water is added. The weight ratio toluene:dimethylformamide:water can be between 1:0.01:2 and 1:0.05:6, preferably it is between 1:0.4:4 and 1:0.05:6. The mixture is stirred for between 5 and 20 hours, preferably between 7 and 10 hours at a temperature between 20° C. and 25° C. to provide a suspension. The suspension is filtered to provide a solid compound (4) that can be dried.

Using the presented process can significantly decrease the reaction time and improve the purity of compound (4) with comparison to the prior art process.

We have also surprisingly found that compound (4) can be purified by a process comprising:
a. Dissolving compound (4) in a mixture comprising toluene and water and a solvent selected from tetrahydrofurane or dimethylformamide or a mixture thereof;
   preferably at a temperature between 60° C. and 110° C.;
b. Isolating solid form of compound (4).

The compound (4) is suspended in the toluene. The weight ratio between compound (4) and the toluene can be between 1:1.8 and 1:5, preferably it is between 1:2 and 1:4. The mixture is warmed to a temperature between 70° C. and 100° C., preferably between 80° C. and 90° C. To the mixture tetrahydrofurane or dimethylfomamide or a mixture thereof is added to dissolve the compound (4). The weight ratio between tetrahydrofurane:toluene or dimethylfomamide:toluene or tetrahydrofurane/dimethylformamide mixture:toluene can be between 1:20 and 1:60, preferably between 1:30 and 1:55, more preferably between 1:40 and 1:50. Water is added to the mixture. The weight ratio toluene:water can be between 1:1 and 1:3, preferably it is between 1:1.2 and 1:1.7. The mixture is stirred at a temperature between 60° C. and 110° C. for between 0.5 and 3 hours. The mixture is cooled to a temperature between 15° C. and 30° C., preferably between 20° C. and 25° C. and the precipitated solid is isolated. The solid compound (4) can be isolated by any suitable method, for example by filtration or by use of centrifuge.

Using the presented purification process can further improve the purity of compound (4) in comparison with the prior art process.

The compound (4) is transformed into compound (1) by a process comprising:
a. Transforming of compound of formula (4) into compound of formula (5):

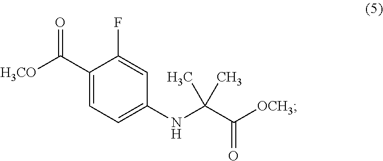 [SMILES: COC(=O)c1ccc(NC(C)(C)C(=O)OC)cc1F]

b. Reacting compound of formula (5) with compound of formula (6) to provide compound of formula (7):

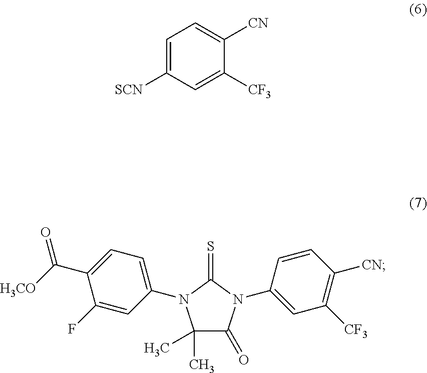

c. Transforming compound of formula (7) into compound of formula (1) or a salt thereof; or
d. Reacting compound of formula (4) with compound of formula (6) to provide compound of formula (7);
e. Transforming compound of formula (7) into compound of formula (1) or a salt thereof.

The transformation can be done by a process known in the prior art, for example CN104803918 or WO2017/081702 application or by following process.

Compound (4) is dissolved in methanol. The concentration of compound (4) in methanol can be between 0.15 g/ml and 0.3 g/ml. To the solution an acid, such as HCl or H₂SO₄ is added. The molar ratio between the acid and compound (4) can be between 1.1.8 and 1:5, preferably it is between 1:2 and 1:3. The mixture is stirred at a temperature between 55° C. and 80° C. for between 10 and 20 hours. The reaction progress can be monitored by a suitable analytical technique, e.g. by HPLC or GC. After the reaction is finished, the mixture is concentrated to approx. 65-70% of the original volume. To the mixture toluene and water are added. The weight ratio between the added toluene and the mixture can be between 0.8:1 and 1.2:1. The weight ratio between the added water and the mixture can be between 0.4:1 and 0.8:1. The mixture is concentrated to approx. ½ of the original volume. The mixture is cooled to between 45° C.-55° C. To the mixture toluene is added. The weight ratio between added toluene and the mixture can be between 0.8:1 and 1.2:1. The layers are separated (at 40-50° C.). To the water layer, other water is added. The weight ratio between the added water and the water was between 1.4:1 and 2:1. To the mixture toluene is added. The weight ratio between added toluene and the added water was 0.8:1 and 1.2:1. The layers are separated (at 40-50° C.). The toluene layers are mixed together. To mixed layers a solution of NaHCO₃ in water is added. The concentration of the solution can be between 0.07 g/ml and 1.5 g/ml. The weight ratio between NaHCO₃ solution and the toluene layers can be between 1:2.5 and 1:5, preferably it is between 1:3 and 1:4. The layers are separated, the organic layer is extracted with water. The weight ratio between water and the organic layer can be between 1:6 and 1:10, preferably it is between 1:7 and 1:9. The layers are separated and the organic layer is concentrated to dryness. To the rest methanol is added. The weight ratio between methanol and compound (4) can be between 0.7:1 and 1:1.3, preferably it is 1:1. The mixture is heated to a temperature between 40° C. and 60° C. To the mixture water is added. The weight ratio between previously added methanol and water can be between 1:0.5 and 1:1, preferably it is between 1:0.6 and 1:0.8. The mixture is cooled to a temperature between −20° C. and 20° C., preferably between −10° C. and 10° C. The solid is isolated and dried to provide compound (5). The solid compound (5) can be isolated by any suitable method, for example by filtration or by use of a centrifuge.

In the subsequent step b. compound (5) is reacted with compound (6) to provide compound (7) in a presence of dimethylsulfoxide. The molar ratio between dimethylsulfoxide and compound (5) can be between 3:1 and 8:1, preferably it is between 3:1 and 5:1. The reaction can be performed in a suitable solvent, for example tetrahydrofurane or toluene or acetonitrile or an acetate, such as isopropylacetate or ethylacetate. Compound of formula (5) is contacted with dimethylsulfoxide, and the mixture can be heated to dissolve compound (5). The mixture can be heated to a temperature between 80° C. and 140° C. Concentration of compound (5) in dimethylsulfoxide can be between 0.5 g/ml and 2 g/ml, preferably it is between 0.7 and 1.5 g/ml. Compound (6) is dissolved in a suitable solvent, for example tetrahydrofurane or toluene or acetonitrile or an acetate, such as isopropylacetate or ethylacetate. Preferably tetrahydrofurane or toluene is used. The concentration of compound (6) in the solvent can be between 0.5 g/ml and 3 g/ml, preferably it is between 0.8 g/ml and 1.5 g/ml. The solution is added in course of 5 or 10 or 20 or 30 or 40 or 50 or 60 minutes to the solution of compound (5). The solution of compound (6) can be also added in several parts, for example in 1 or 2 or 3 or 4 or 5 parts. The mixture is stirred for between 10 and 60 minutes. The mixture is then heated to a temperature between 80° C. and 140° C. and stirred at this temperature for between 1 and 10 hours, preferably between 2 and 5 hours. The reaction progress can be monitored by a suitable analytical technique, e.g. by HPLC or GC. After the reaction is finished, the mixture is cooled to a temperature between 40° C. and 55° C. To the mixture a solution of NaCl in water is added. The concentration of NaCl solution can be between 0.15 g/ml and 0.3 g/ml, preferably it is between 0.18 and 0.25 g/ml. The volume ratio between added NaCl solution and the solvent used in the reaction can be between 1:0.5 and 1:1.3, preferably it is between 1:0.7 and 1:1. To the mixture tetrahydrofurane is added. The volume ratio between added NaCl water solution and tetrahydrofurane can be between 1:1.5 and 1:3, preferably it is between 1:1.8 and 1:2.5. The phases are separated. Water layer is extracted with tetrahydrofurane, the volume ratio between tetrahydrofurane and the water phase can be between 1.8:1 and 3:1, preferably it is between 2:1 and 2.7:1. Tetrahydrofurane phases were mixed together and distilled off to approx. ½ of the original volume. To the mixture ethanol is added. The weight ratio between ethanol and the mixture can be between 1:0.5 and 1:1. The mixture is distilled off to approx. ½ of the original volume. The suspension is cooled to a temperature between −10° C. and 10° C., preferably between −5° C. and 5° C. and stirred at this temperature for between 8 and 15 hours. The solid is isolated and dried to provide compound (7). The solid compound (7) can be isolated by any suitable method, for example by filtration or by use of a centrifuge.

Compound (7) can be further purified by contacting with a mixture of acetone and ethanol preferably under a protective atmosphere (for example nitrogen or argon). The weight ratio between acetone and ethanol can be between 1:2 and 1:6, preferably between 1:3 and 1:4. The weight ratio between compound (7) and acetone can be between 1:0.8 and 1:1.3, preferably it is between 1:1 and 1:1.1. Compound (7) is mixed with acetone. The resulting mixture can be optionally filtered to remove non-dissolved solid. To the filtrate ethanol is added. The mixture is distilled off to approx. 80% (vol %) of the original volume. The suspension was cooled to a temperature between −10° C. and 10° C., preferably between −5° C. and 5° C. and stirred at this temperature for between 2 and 5 hours. The solid is isolated and dried to provide compound (7). The solid compound (7) can be isolated by any suitable method, for example by filtration or by use a centrifuge.

Using the presented purification process can further improve the purity of compound (7) in comparison with the prior art process.

Compound (7) is transformed into compound (1) in the step c. for example by reacting with water solution of methylamine, for example 40% solution can be used. The water solution of methylamine is cooled to a temperature between −30° C. and 0° C., preferably between −20° C. and −10° C. The solution is added to a mixture of compound (7) in a suitable solvent such as tetrahydrofurane. The concentration of compound (7) in the solvent can be between 0.5 g/ml and 2 g/ml, preferably between 0.7 g/ml and 1 g/ml. Or compound (7) can be added as solid to the methylamine water solution. Compound (7) and methylamine are mixed in a course of between 30 and 120 minutes. The molar ratio between the compound (7) and the methylamine can be between 1:18 and 1:50, preferably it is between 1:22 and 1:25. The mixture is stirred at a temperature between −30° C. and 0° C., preferably between −20° C. and −10° C. for between 3 and 10 hours, preferably between 4 and 6 hours. The reaction progress can be monitored by a suitable analytical technique, e.g. by HPLC or GC. After the reaction is finished, compound (1) can be isolate for example by distilling off the solvent or a procedure disclosed in the prior art or by following procedure. To the reaction mixture alcohol such as methanol or ethanol or 1-propanol or isopropanol or butanol or isobutanol or 2-butanol or a mixture thereof is added. Preferably a mixture of 1-propanol and 2-butanol is used. The weight ratio between 1-propanol and 2-butanol can be between 1:0.1 and 0.1:1, preferably it is between 1:0.8 and 1:1.2. The weight ratio between the added alcohol or the mixture and the reaction mixture can be between 1:1 and 1:3, preferably between 1:1 and 1:5. The mixture is then distilled off to ⅓ of the original volume under a protective atmosphere (for example argon or nitrogen) to provide a suspension that is cooled to a temperature between −20° C. and 20° C., preferably between 0° C. and 20° C. and is stirred at this temperature for between 30 and 120 minutes. The obtained solid compound (1) is isolated and dried. The solid compound (1) can be isolated by any suitable method, for example by filtration or by use of a centrifuge. The isolated solid compound (1) is preferably Form A disclosed in WO2014/043208.

Compound (4) can be alternatively transformed directly into compound (7) and subsequently compound (7) into compound (1) as disclosed in WO2017/081702 application.

We have also surprisingly found that compound (1) can be transformed into a solid form of 1,4-dioxane solvate. The form can be characterized by XRPD pattern having 20 values 13.2°, 17.5°, 20.8° 2θ (±0.2 degrees 2θ). The form can be further characterized by XRPD pattern having 20 values 10.7°, 11.4°, 13.2°, 17.5°, 20.8° 2θ (±0.2 degrees 2θ). The form can be further characterized by 2θ values (±0.2 degrees 2θ) stated in following table:

| Angle | Intensity |
|---|---|
| 2-Theta ° | % |
| 5.8 | 18.4 |
| 9.6 | 4.8 |
| 10.2 | 4.6 |
| 10.7 | 46.5 |
| 10.9 | 12.9 |
| 11.4 | 59.6 |
| 11.6 | 55.1 |
| 13.2 | 60.4 |
| 13.9 | 7.7 |
| 14.3 | 19.2 |
| 14.8 | 46.2 |
| 15.4 | 34.0 |
| 15.8 | 28.5 |
| 16.8 | 31.4 |
| 17.1 | 15.0 |
| 17.5 | 51.4 |
| 19.4 | 29.8 |
| 19.7 | 10.4 |
| 19.9 | 20.1 |
| 20.3 | 5.9 |
| 20.8 | 100.0 |
| 21.4 | 15.4 |
| 22.0 | 41.4 |
| 22.2 | 21.2 |
| 22.5 | 15.6 |
| 22.6 | 22.2 |
| 23.0 | 23.2 |
| 23.5 | 48.7 |
| 24.1 | 24.8 |
| 24.5 | 17.8 |
| 24.7 | 28.9 |
| 25.0 | 32.4 |
| 25.4 | 7.5 |
| 25.7 | 6.7 |
| 26.1 | 9.6 |
| 26.7 | 6.1 |
| 27.0 | 3.0 |
| 27.7 | 6.5 |
| 28.1 | 4.3 |
| 28.8 | 12.5 |
| 29.0 | 20.8 |
| 29.4 | 11.2 |
| 30.1 | 12.3 |
| 30.7 | 12.7 |
| 31.1 | 5.0 |
| 31.2 | 4.3 |
| 31.6 | 6.7 |
| 31.9 | 7.8 |
| 32.3 | 5.5 |
| 32.7 | 4.0 |
| 33.3 | 8.8 |
| 33.6 | 4.1 |
| 33.9 | 6.8 |
| 34.1 | 3.6 |
| 34.7 | 6.0 |

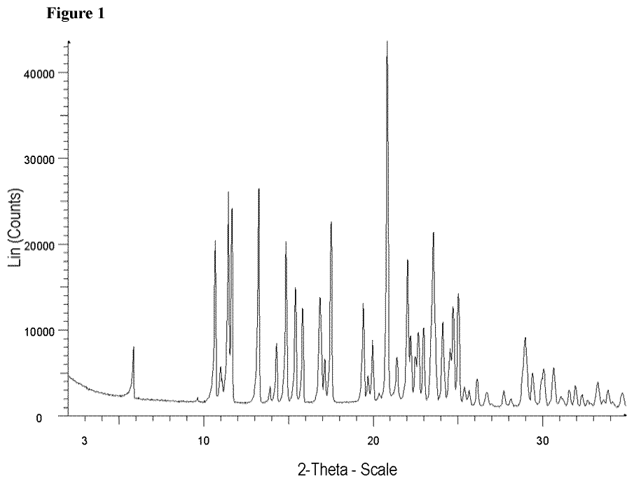

The solid form can be also characterized by XRPD pattern depicted in FIG. 1.

The 1,4-dioxane solvate of compound (1) can be prepared by a process comprising:
a. dissolving of compound of formula (1) with 1,4-dioxane;
b. isolating 1,4-dioxane solvate of compound of formula (1).

The concentration of compound (1) in 1,4-dioxane can be between 1.5 g/ml and 3.5 g/ml, preferably it is between 2 g/ml and 3 g/ml. The mixture is heated to a temperature between 60° C. and 120° C., preferably between 90° C. and 110° C. to dissolve the compound (1). The mixture was cooled to a temperature between −30° C. and 30° C., preferably between 10° C. and 25° C. and stirred at this temperature for between 30 and 120 minutes. 1,4-dioxane solvate of compound (1) can be also isolated by addition of an antisolvent to the mixture of compound (1) in 1,4-dioxane. 1,4-dioxane solvate isolated and dried. The solid 1,4-dioxane solvate of compound (1) can be isolated by any suitable method, for example by filtration or by use of centrifuge.

1,4-dioxane solvate of compound (1) can be used for purification of compound (1). In this case the 1,4-dioxane solvate is transformed to a solid form of compound (1) by a process known from the prior art or above described procedure using an alcohol or a mixture of alcohols. The isolated solid compound (1) is preferably Form A disclosed in WO2014/043208.

The invention will be further illustrated by the following examples.

### EXAMPLES

XRPD spectrum of solid compounds was obtained using the following measurement conditions:

Panalytical Empyrean diffractometer with Θ/2Θ geometry (transmition mode), equipped with a PixCell 3D detector:

| | |
|---|---|
| Start angle (2θ): | 2.0° |
| End angle (2θ): | 35.0° |
| Step size: | 0.026° |
| Scan speed: | 0.0955°/seconds |
| Radiation type: | Cu |
| Radiation wavelengths: | 1.5406Å (Kα1), primary monochromator used |
| Divergence slit: | 1/2° |
| Antiscatter slit: | 1/2° |
| Soller slit: | 0.02 rad |
| Detector slit: | 7.5 mm |
| Rotation speed: | 30 rpm |

### Example 1: Preparation of 2-((3-fluoro-4-(methoxycarbonyl)phenyl)amino)-2-methylpropanoic acid, Compound (4)

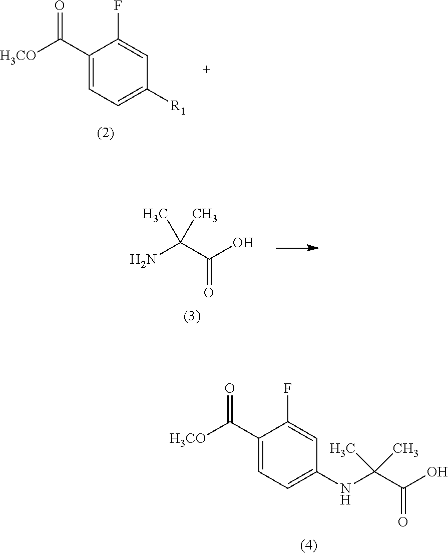

150 g of compound (2), 93 g of compound (3), 225 g of K₂CO₃ and 12 g of CuCl were suspended in 123 g of dimethylformamide (DMF) and 29 g of water at 25° C. The mixture was stirred and heated under a protective atmosphere of nitrogen at 110° C. for 2.5 hrs. The nitrogen was streamed above the reaction mixture at a rate 30-40 l/min. The suspension was cooled to 20-25° C. and diluted with 900 g of water and 90 g of tetrahydrofurane. pH of the mixture was adjusted to 2.0-2.2 using 36% water solution of hydrochloric acid. The mixture was extracted with a mixture of 300 g of toluene and 150 g of tetrahydrofurane at 25-30° C. The phases were separated and water phase was extracted with 200 g of toluene. The phases were separated and water phase was extracted with 100 g of toluene. The combined toluene phases were extracted with 150 g of water. Phases were separated and the toluene phase was distilled off to total amount of 270 g. A mixture of 300 g of toluene, 10 g of DMF and 750 g of water was added. The mixture was stirred for 14 hours to provide a suspension. The suspension was filtered at 20° C. and washed mixture of 180 g of water and 80 g of toluene. to provide 153 g of compound (4) as wet crystals that were drying to provide 122 g of compound (4).

### Example 2: Purification of Compound (4)

562.6 g of compound (4) (purity 95%, HPLC IN) was suspended at 20-25° C. in 2000 g of toluene. The suspension was stirred and heated to 80° C. 50 g of dimethylformamide was added to dissolve the suspension. To the solution 2800 g of water was added. The mixture was stirred for 3 hours to provide a suspension. The mixture was filtered and washed with 100 g of toluene and 200 g of water. The filtered mass was dried in the drier (65° C., 100 mbar, N₂ bleed, 2 hrs) to provide 252 g of crystalline compound (4) (76.69% of theory) as white crystals having 98.2% purity (HPLC IN).

### Example 3: Preparation of Enzalutamide, Compound (1)

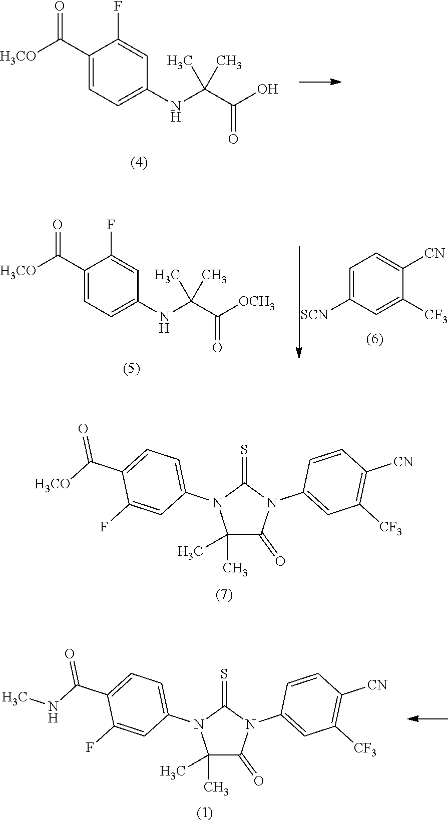

### Example 3a: Preparation of methyl 2-fluoro-4-((1-methoxy-2-methyl-1-oxopropan-2-yl)amino)benzoate, compound (5)

330 g of compound (4) was mixed with 1320 g of methanol. 264 g of sulphuric acid (96-98%) was added. The solution was heated to 65° C. and stirred at this temperature for 15 hours. The reaction mixture was distilled off to a total amount of 1081 g. To the mixture 660 g of water and 1000 g of toluene were added. The mixture was distilled off to ½ of the original volume. To the mixture 848 g of toluene at 50° C. was added. Layers were separated. To the water layer 1056 g of water and 590 g of toluene were added. Layers were separated. To the water layer 264 g of water and 264 g of toluene were added. Layers were separated. To the combined toluene layers a water solution of sodium hydrogencarbonate (58.3 g of NaHCO₃ in 555 g of water) was added. Layers were separated. The organic layer was washed with 264 g of water. Organic layer was concentrated to dryness. The rest was suspended in 317 g of methanol and the mixture was heated to 50° C. 238 g of water was added. The suspension was cooled to 5° C. The solid was filtered and washed with mixture of 79.2 g of methanol and 66 g of water, dried in the drier (65° C., 100 mbar, N₂, 2 hours) to provide 300 g of compound (5) (86.17% of theory) of crystalline compound (5), purity 99.4% (UPLC).

The compound (5) can be purified by following process.

15 g of compound (5) having purity 99.4% was suspended in 15 g of methanol and the mixture was heated to 65° C. to dissolved the compound (5). 11.4 g of water was added. The suspension was cooled to 5° C. The solid was filtered off and washed with a mixture of 4 g of methanol (4 g) and 3 g of water, dried in the drier (65° C., 100 mbar, N₂, 2 hours) to provide 14.60 g (97.33% of theory) of crystalline compound (5), purity 99.8% (UPLC).

### Example 3b: Preparation of methyl 4-(3-(4-cyano-3-(trifluoromethyl)phenyl)-5,5-dimethyl-4-oxo-2-thioxoimidazolidin-1-yl)-2-fluorobenzoate, Compound (7)

190 g of compound (5) was suspended in 250 g of dimethylsulfoxide (DMSO). The mixture was heated to 120° C. to dissolve the compound (5). 285 g of compound (6) was mixed with 200 g of tetrahydrofurane. To limit the risk for using tetrahydrofurane at high temperatures toluene can be used as a solvent instead. The solution of compound (6) was added in the course of 20 minutes into the mixture if compound (5) in dimethylsulfoxide. The mixture was stirred for 30 minutes and a solution of 47.5 g of compound (6) in 30 g of tetrahydrofurane was added in the course of 10 minutes. The reaction mixture was heated to 120° C. and stirred at this temperature for 3 hours. The mixture was cooled to 50° C. A solution of 114 g of NaCl in 570 g of water was added. To the mixture 1000 g of tetrahydrofurane was added and layers were separated. The water layer was extracted with 200 g of tetrahydrofurane. Mixed organic phases were distilled off to overall mass of 800 g. To the mixture 1110 g of ethanol was added. The mixture was distilled off to overall mass of 1160 g. The suspension was stirred overnight at 0° C. Solid mass was filtered and washed with 2×50 g of ethanol. The filter cake was dried in the drier (65° C., 100 mbar, N₂, 3 hours) to provide 286.72 g of compound (7) (87.31% of theory) having purity 95.5% (UPLC).

The compound (7) can be purified by following process.

513 g of compound (7) was suspended in 450 g of acetone at 25° C. and stirred at this temperature for 30 minutes. The mixture was filtered off, washed with 60 g of acetone. The mother liquor was mixed with 1900 g of ethanol. The mixture was distilled off at 70° C. to overall mass of 1950 g. The suspension was cooled to 10° C. in the course of 2 hours. Solid mass was filtered off, washed with 2×140 g of ethanol and dried in the drier (65° C., 100 mbar, N₂, 2 hours) to provide 475.80 g (92.72% of theory) of compound (7) having purity 99.9% (UPLC).

### Example 3c: Preparation of Enzalutamide, Compound (1)

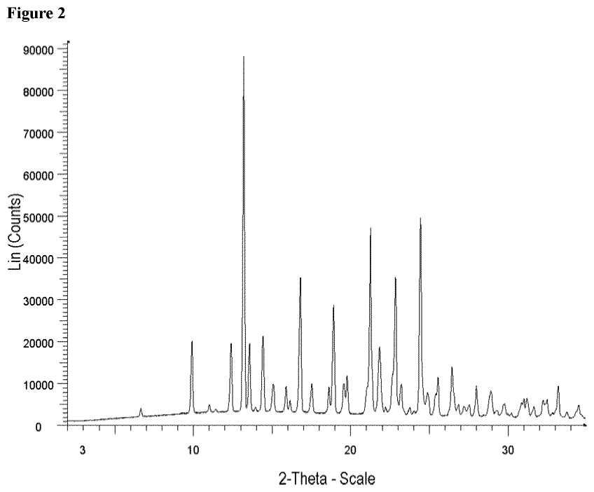

1000 g of 40% solution of methylamine in water was cooled to (−13° C.). To this solution a solution of 250 g of compound (7) in 300 g of tetrahydrofurane was added in the course of 60 minutes. The mixture was stirred for 4 hours. To the mixture a mixture of 625 g of 1-propanol and 625 g of 2-butanol was added. The mixture was cooled to 0° C. and filtered. The mixture was particularly distilled off to approx. ⅓ of original weight with nitrogen treatment under vacuum at 0-20° C. The suspension was cooled to 20° C. and stirred at this temperature for 1 hour. The solid was filtered off, washed with a mixture of 50 g 1-propanol and 50 g of 2-butanol, dried in the drier (65° C., 100 mbar, N₂, 5 hours) to provide 220 g (88.18% of theory) of compound (1) having purity 99.9%. XPRD pattern of obtained solid corresponds to XRPD pattern depicted in FIG. 2.

### Example 4: Preparation of 1,4-dioxane Solvate of Compound (1)

10 g of Enzalutamide, compound (1) was suspended at 20-25° C. in 4 g of 1,4-dioxane and suspension was stirred and heated to 100° C. and stirred at this temperature for 15 minutes. Suspension was cooled to 25° C. during 60 min. The solid was filtered and washed with 1 ml of 1,4-dioxane. The filtered solid was dried in the drier (80° C., 100 mbar, N₂, 24 hours) to provide 9.41 g (85% of theoretical yield) of 1,4-dioxane solvate of compound (1), purity 99.7% (UPLC).

1,4-dioxane solvate of compound (1) can be transformed into a solid form of compound (1) by following process. 9.41 g of 1,4-dioxane solvate was mixed with a mixture of 26 g of 1-propanol and 26 g of 2-butanol was added. The mixture was cooled to 0° C. and filtered. The mixture was particularly distilled off to approx. ½ of the original weight with nitrogen treatment under vacuum at 0-20° C. The suspension was cooled to 20° C. and stirred at this temperature for 1 hour. The solid was filtered off, washed with a mixture of 5 g 1-propanol and 5 g of 2-butanol, dried in the drier (65° C., 100 mbar, N₂, 5 hours) to provide solid compound (1) in the yield 95%. XPRD pattern of obtained solid corresponds to XRPD pattern depicted in FIG. 2.

1. A process for preparation of compound of formula (1) or a salt thereof comprising: 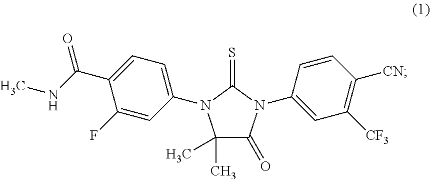 [SMILES: CNC(=O)c1ccc(N2C(=S)N(c3ccc(C#N)c(C(F)(F)F)c3)C(=O)C2(C)C)cc1F]
a) reacting compound of formula (2) with compound of formula (3) in a solvent under a protective atmosphere, wherein the protective atmosphere is an inert gas streamed above the reaction mixture to provide compound (4) 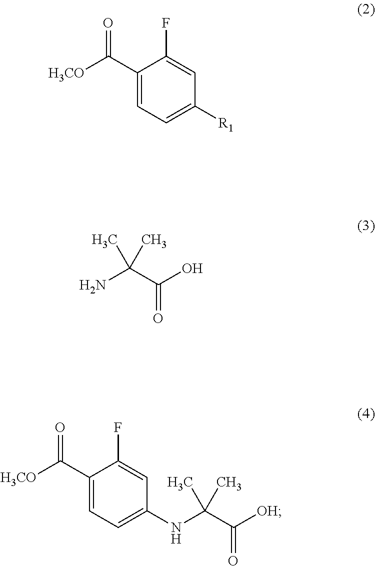
R₁ is a leaving group; and
b) transforming compound (4) into compound (1).

2. The process according to claim 1 wherein R₁ is a halogen or alkyl or aryl toluene sulfonate or perfluoroalkylsulfonate.

3. The process according to claim 1 wherein the inert gas is selected from nitrogen or argon.

4. The process according to claim 1 wherein the inert gas in streamed above the reaction mixture at a rate between 1 and 100 l/minute.

5. The process according to claim 4 wherein the rate is between 10 and 100 l/minute.

6. The process according to claim 5 wherein the rate is between 30 and 60 l/minute.

7. A process for purification of compound of formula (4) comprising:
a) dissolving compound (4) 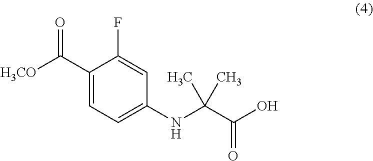 [SMILES: COC(=O)c1ccc(NC(C)(C)C(=O)O)cc1F]
in a mixture comprising toluene and water and a solvent selected from tetrahydrofurane or dimethylformamide or a mixture thereof; and
b) isolating solid form of compound (4).

8. The process according to claim 7 wherein the ratio (wt:wt) between toluene:solvent selected from tetrahydrofurane or dimethylformamide or a mixture thereof is between 1:20 and 1:60.

9. The process according to claim 8 wherein the ratio is between 1:30 and 1:55 (wt:wt).

10. The process according to claim 7 wherein the ratio (wt:wt) toluene:water is between 1:1 and 1:3.

11. The process according to claim 10 wherein the ratio (wt:wt) is between 1:1.2 and 1:1.7.

12. (canceled)

13. (canceled)

14. The process according to claim 1 wherein the transforming step comprises:
(I) the steps comprising
a) transforming of compound of formula (4) into compound of formula (5): 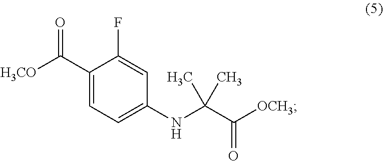 [SMILES: COC(=O)c1ccc(NC(C)(C)C(=O)OC)cc1F]
b) reacting compound of formula (5) with compound of formula (6) to provide compound of formula (7): 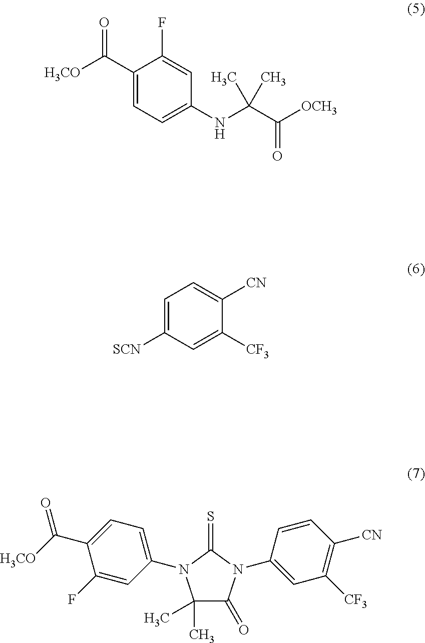
and
c) transforming compound of formula (7) into compound of formula (1) or a salt thereof; or
(II) the steps comprising
d) reacting compound of formula (4) with compound of formula (6) to provide compound of formula (7); and
e) transforming compound of formula (7) into compound of formula (1) or a salt thereof.

15. A process for purification of compound of formula (7) comprising contacting compound (7) 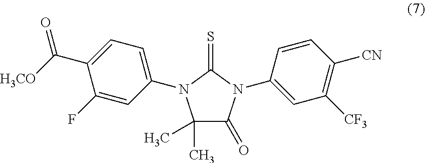 [SMILES: COC(=O)c1ccc(N2C(=S)N(c3ccc(C#N)c(C(F)(F)F)c3)C(=O)C2(C)C)cc1F]
with acetone and ethanol.

16. The process according to claim 15 wherein the weight ratio between acetone and ethanol is between 1:2 and 1:6.

17. The process according to claim 16 wherein the weight ratio between acetone and ethanol is between 1:3 and 1:4.

18-24. (canceled)
